(19) United States
(12) Patent Application Publication
(10) Pub. No.: US 2014/0060537 A1
(43) Pub. Date: Mar. 6, 2014

(54) DEVICE FOR THE MECHANICAL RESPIRATION OF A PATIENT AND PROCESS FOR THE HYGIENIC PROCESSING THEREOF

(71) Applicants: Hans-Ullrich HANSMANN, Barnitz (DE); Michael RIECKE, Lubeck (DE); Henryk SCHNAARS, Lubeck (DE)

(72) Inventors: Hans-Ullrich HANSMANN, Barnitz (DE); Michael RIECKE, Lubeck (DE); Henryk SCHNAARS, Lubeck (DE)

(73) Assignee: DRAGER MEDICAL GMBH, Lubeck (DE)

(21) Appl. No.: 13/774,111

(22) Filed: Feb. 22, 2013

(30) Foreign Application Priority Data
Feb. 23, 2012 (DE) 10 2012 003 549.7

(51) Int. Cl. A61L 2/20; A61M 16/22; A61M 16/00

(52) U.S. Cl. CPC A61L 2/20; A61M 16/00; A61M 16/22
USPC 128/204.17; 128/204.18; 128/205.12; 422/28

(57) ABSTRACT

A device (10) for the mechanical respiration of a patient (12) has a gas delivery device (14), which includes a flow module (16) for generating a gas stream (A), a gas outlet (24) and a gas inlet (34). The device (10) includes a gas-carrying short-circuiting unit (56), by which the gas outlet (24) and the gas inlet (34) can be coupled with one another to short-circuit the gas delivery device (14), and a plasma generator (62) coupled into the short-circuited gas delivery device (14) for enriching the gas stream (A) generated in the short-circuited gas delivery device (14) with a disinfecting plasma.

### CROSS REFERENCE TO RELATED APPLICATIONS

This application claims the benefit of priority under 35 U.S.C. §119 of German Patent Application DE 10 2012 003 549.7 filed Feb. 23, 2012, the entire contents of which are incorporated herein by reference.

### FIELD OF THE INVENTION

The present invention pertains to a device for the mechanical respiration (also known as ventilation) of a patient with a gas delivery device, which comprises a flow module for generating a gas stream, a gas outlet and a gas inlet as well as to a process for the hygienic processing of a device intended for the mechanical respiration of a patient.

### BACKGROUND OF THE INVENTION

Breathing systems, which are to be processed hygienically before changing over from one patient to another, are used in anesthesia and respiration therapy. Thus, the breathing system must be freed of microorganisms that are present in the gas expired by a patient. Sterility of the breathing system must be achieved in case of infectious patients.

Common processing methods are, for example, steam autoclaving or treatment with bactericidal substances such as ethylene oxide and formaldehyde. The use of such substances has considerable drawbacks. Thus, environmental pollution caused by these substances cannot be ruled out. There also is a risk of contamination of the breathing gas produced later by the breathing system unless predefined process parameters are complied with accurately during the hygienic processing. Finally, these substances also represent a great burden for the materials used in the device components to be processed.

Furthermore, processing methods that utilize the disinfecting action of a so-called cold plasma, which is produced under atmospheric pressure conditions, have come into use recently. A plasma generator for producing such a disinfecting plasma is known, for example, from EP 2 223 704 A1. The use of plasma for the hygienic processing of respirators is described in DE 10 2004 024 175 A1. Furthermore, reference is made for the state of the art to DE 10 2009 028 190 A1, which likewise deals with the use of plasma for hygienic processing.

It is common to all the methods known hitherto that processing means provided specifically for this purpose must be made available, which are located, as a rule, in a central location, in order to disinfect a plurality of medical devices. This has a considerable drawback in terms of the time required, handling and costs. For example, the availability of the devices to be processed is considerably reduced if only a single processing means or a few processing means are available in a hospital. It is also frequently desirable to process individual device components only. In this case, these must be removed at first from the medical device and then introduced into the processing means. This is comparatively complicated as well.

A device for disinfecting the interior of an apparatus for respirating a patient is known from DE 10 2009 016 542 A1. The device is connected to the apparatus and can use a flow module of the apparatus in order to generate a disinfecting gas stream, which flows through the apparatus. The disinfecting gas stream contains, among other things, ozone, which is generated with an ozone generator, for example, an amalgam-doped, low-pressure mercury vapor radiator.

A sterilizing chamber for sterilizing various objects with the use of a plasma, which is enriched with a gas and is generated by a plasma generator, is described in US 2007/0207054 A1.

U.S. Pat. No. 5,810,002 discloses a respirator for a patient with a gas delivery device, which has a flow module, a gas inlet and a gas outlet.

### SUMMARY OF THE INVENTION

An object of the present invention is to make possible a reliable, simple hygienic processing of a device intended for the mechanical respiration of a patient under gentle conditions for the device.

The device according to the present invention comprises a gas delivery device, which has a flow module for generating a gas stream as well as a gas outlet and a gas inlet. The gas delivery device is short-circuited for the purpose of hygienic processing. The device has a gas-carrying short-circuiting unit for this, by which the gas outlet and the gas inlet can be coupled with one another. A preferably microstructured plasma generator, which is capable of generating, in a manner known per se, a disinfecting plasma, with which the gas stream generated by the flow module is enriched, is coupled according to the invention into the gas delivery devices short-circuited in this manner. The conduction of the process and the validation of the processing can be controlled by the device itself. The plasma is preferably generated as a so-called cold plasma under atmospheric pressure conditions. The ionized gas molecules forming the plasma have, for example, a life in the range of 10 sec to 30 sec. During this life, they are able to kill microorganisms in the gas delivery device.

Consequently, the present invention utilizes the gas flow generated by the flow module, which is used to respirate (also known as ventilate) the patient during normal operation, as a transport medium for the disinfecting plasma during the processing operation. It is ensured hereby that the disinfecting plasma will reach exactly the areas that had also been reached before by the breathing gas possibly contaminated with microorganisms during the normal operation.

The advantageous utilization of the flow module to distribute the plasma in the gas delivery means makes possible a self-disinfection operation, which considerably simplifies the hygienic processing of the respirator or anesthesia device. The user is thus relieved of complicated processing operations by this self-disinfection operation.

Due to the fact that the present invention utilizes device components such as the flow module, which are already present in the device anyway, for the hygienic processing, the amount of hardware needed is also markedly lower than before. Another advantage is that the conduct of the process and the validation of processing can be controlled and documented by the device itself. This control present in the device ensures accurate compliance with a defined processing process, which is characterized by process parameters determined in advance, such as flow rates and operating times. Thus, it is possible, for example, to detect and document a manual intervention with the course of the process.

Compared to the hitherto known processing means, the device according to the present invention has, moreover, the advantage that the device components to be processed do not have to be removed from the device. The availability of the device is thus restored in a short time after the processing thereof.

The present invention can be applied to rebreathing systems and non-rebreathing systems alike. The gas outlet of the gas delivery device is associated in both cases with the inspiration branch and the gas inlet of the gas delivery device is associated with the expiration branch of the system.

It is advantageous especially in case of a rebreathing system to release part of the gas stream, which forms a closed circuit in the short-circuiting operation according to the present invention from the breathing system, for example, through an anesthetic gas escape line and to replace it with dry fresh gas. The percentage of fresh gas is, for example, in a range of 5% to 50%. It may also be controlled intermittently between 0% and 100%. The pressure fluctuation generated hereby in the short-circuited gas delivery device ensures turbulences, by which the disinfecting action of the plasma is supported.

It is possible in case of, e.g., a non-rebreathing system used in respiration therapy to rinse the device components, e.g., an expiration valve and an expiration volume flow sensor, with the disinfecting plasma.

The plasma may be sent over an activated carbon filter in both a rebreathing system and a non-rebreathing system in order to remove ions and radicals from the waste air generated.

In a preferred embodiment, the plasma generator is integrated in the short-circuiting unit. If the short-circuiting unit is a component that is separate from the gas delivery device and is coupled with the gas outlet and the gas inlet of the gas delivery device in the self-disinfection operation only, this short-circuiting unit may be used with the plasma generated integrated in it for the hygienic processing of a plurality of respirators.

The short-circuiting unit is, for example, a gas-carrying stub line, which connects the gas inlet to the gas inlet of the gas delivery device. The short-circuiting unit preferably comprises a controllable short-circuiting valve for opening the stub line. This embodiment is especially advantageous when the short-circuiting unit is integrated in the respirator. The short-circuiting valve is used in this case to switch the short-circuiting unit into the active mode in the self-disinfection operation.

The stub line may be formed from a tube with two connection parts, of which a first connection part is coupled with the gas outlet and a second connection part with the gas inlet. In this variant, the short-circuiting unit is a component that is provided separately from the gas delivery device and is connected to the respirator in the self-disinfection operation only.

The short-circuiting unit may also comprise a Y-piece with three connection parts, of which a first connection part can be coupled with the gas inlet, a second connection part with the gas inlet and a third connection part with the plasma generator. Such a Y-piece is usually used in breathing systems for the separation in space of inspiration gas from expiration gas. The short-circuit of the gas delivery device provided in the self-disinfection operation is embodied in a simple manner by coupling the plasma generator with the connection part that is connected with the patient in the respiration operation. This connection part of the Y-piece is not absolutely coupled directly with the processing here. It is possible, moreover, that further gas-carrying device components, such as water traps, tubes and/or filters, are located with the Y-piece and the plasma generator and are thus rinsed with the disinfecting plasma during the short-circuiting operation.

The gas delivery device preferably has a mechanical interface for optionally coupling a gas-carrying device component or the plasma generator. A gas-carrying device component, e.g., a carbon dioxide absorber, is coupled into the gas delivery device via this mechanical interface in normal respiration operation. To process the device hygienically, the plasma generator is then coupled instead of said device component.

In an especially preferred embodiment, the gas delivery device has a heater for heating the gas stream enriched with the disinfecting plasma. This is especially advantageous when applying the present invention to a rebreathing system in order to dry the gas stream circulating in the gas delivery device.

The flow module is preferably a blower, e.g., a radial blower, or a piston and cylinder unit. If the gas outlet and gas inlet are located on a flow module, the flow module itself is short-circuited by installing the short-circuiting unit and is rinsed with disinfecting plasma by the coupled plasma generator.

The short-circuiting unit is preferably formed from a blower, in which the plasma generator is arranged and which has a housing inlet that can be coupled with the gas outlet of the flow module and a housing outlet that can be coupled with the gas inlet of the flow module. It is possible due to this variant to hygienically process a plurality of flow modules with one and the same short-circuiting unit.

The present invention provides, furthermore, for a process for the hygienic processing of a device intended for the mechanical respiration of a patient. In an especially preferred embodiment of this process, the plasma generator is put cyclically into operation. It was thus found that cyclic operation of the plasma generator makes possible a highly effective and energy-saving disinfection of the device.

The present invention will be described below on the basis of exemplary embodiments with reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

FIG. 1 is a schematic view showing a first exemplary embodiment of the device according to the present invention in the respiration operation;

FIG. 2 is a schematic view showing the device according to the first exemplary embodiment in the self-disinfection operation;

FIG. 3 is a schematic view showing a second exemplary embodiment of the device according to the present invention in the respiration operation;

FIG. 4 is a schematic view showing the device according to the second exemplary embodiment in the self-disinfection operation;

FIG. 5 is a schematic view showing a third exemplary embodiment of the device according to the present invention in the self-disinfection operation;

FIG. 6 is a schematic view showing a fourth exemplary embodiment of the device according to the present invention in the self-disinfection operation; and

FIG. 7 is a schematic view showing a fifth exemplary embodiment of the device according to the present invention in the self-disinfection operation.

### DESCRIPTION OF THE PREFERRED EMBODIMENTS

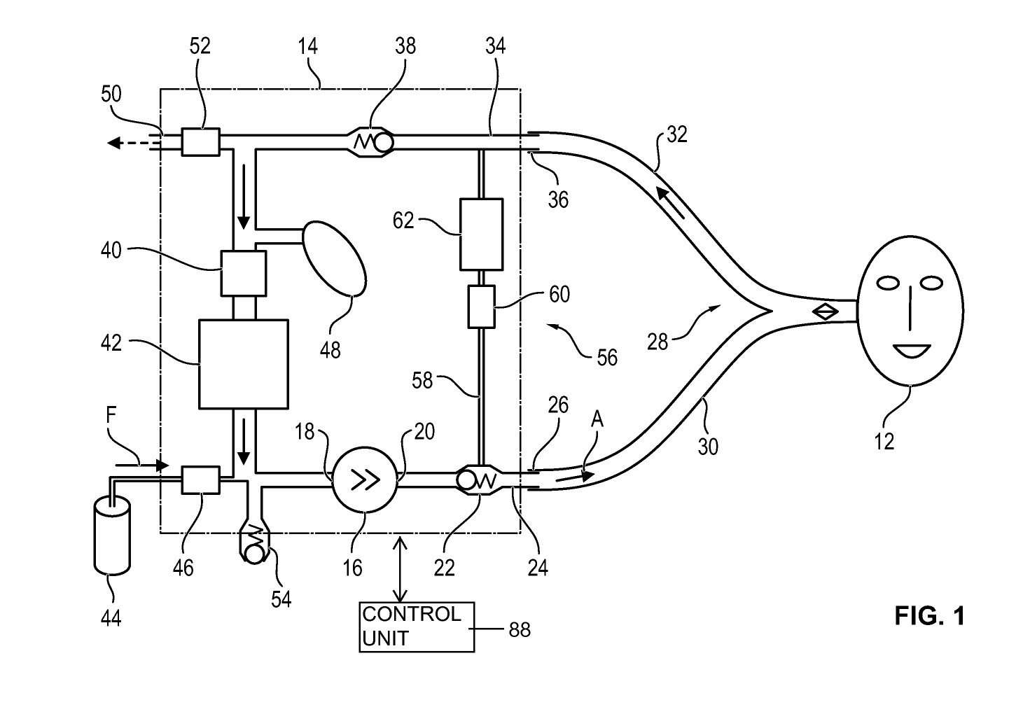

Referring to the drawings in particular, FIG. 1 shows a device 10 according to the present invention as a first exemplary embodiment. The device 10 forms a rebreathing system used in anesthesia, which is used for the controlled respiration of a patient 12. Device 10 supplies the patient 12 with breathing gas, to which anesthetic gases are added.

Device 10 comprises a gas delivery device, which is generally designated by 14 in FIG. 1 and is formed from the device components explained in more detail below, which carry the breathing gas inspired and expired by patient 12. The normal respiration operation of the device 10 shall be described first with reference to FIG. 1.

Gas delivery device 14 contains a radial blower 16, which is operated by means of a control unit 88 (the control unit 88 is operatively connected to gas delivery device 14 as shown only in FIG. 1), such that a pressure that is higher than the pressure at the gas inlet 18 of blower 16 builds up at the gas outlet 20 thereof.

In an inspiration cycle, the breathing gas stream A reaches via an opened inspiration nonreturn valve 20 a gas outlet 24, to which a first connection part 26 of a Y-piece 28 is attached. The breathing gas stream A thus enters an inspiration section 30 of the Y-piece 28 and reaches patient 12. As a consequence of the pressure built up by the radial blower 16, the breathing gas is pressed into the lungs of patient 12.

In an expiration cycle following the inspiration cycle, during which the radial blower 16 reduces the pressure admitted to the gas delivery device 14, the breathing gas expired by patient 12 enters, via an expiration section 32 of Y-piece 28, a gas inlet 34 of the gas delivery device 14, at which the aspiration section 32 is arranged via a second connection part 36. An expiration valve 38 is opened in the expiration cycle, so that the breathing gas stream A is passed on in the gas delivery device 14 and is heated by a heater 40 in the process in order to reduce condensation in the breathing gas stream. The breathing gas stream A is then sent back through a carbon dioxide absorber 42 to the radial blower 16. The carbon dioxide absorber 42 is, for example, a container filled with breathing lime.

The carbon dioxide absorber 42 binds the carbon dioxide, which is contained in the breathing gas, as a rule, at concentrations of about 4 vol. % to 5 vol. %. To compensate this loss of volume flow, a fresh air feed device 44 is provided, which feeds fresh air F (possibly with anesthetic gases added) into the gas delivery device 12 via a fresh air valve 46. A vacuum caused by the filtering out of carbon dioxide in the gas delivery device 14 is thus prevented from building up.

For reasons of safety, the gas delivery device 14 is operated with a slightly positive volume balance in respect to the fresh air F fed in. This means that the volume of the fresh air F fed into the gas delivery device 14 is somewhat larger than the volume of the carbon dioxide that the carbon dioxide absorber 42 removes from the breathing gas stream. A manual bag 48, in which a pressure builds up corresponding to the volume flow excess caused by the fresh air feed, is used to take this positive volume flow balance into account.

If the volume flow excess is so large that it cannot be compensated by the manual bag 48, a valve 52 arranged in an anesthetic gas escape line 50 opens, as a result of which the gas excess escapes into the environment.

The device 10 has, furthermore, a room air emergency valve 54, which opens in an emergency, in which the pressure in the gas delivery device 14 drops below the atmospheric pressure and thus it draws room air into the gas delivery device 14.

The device 10 has, finally, a short-circuiting unit, generally designated by 56 in FIG. 1, which is used for the self-disinfection operation of the device 10, which will be described in more detail below. The short-circuiting unit 56 is formed from a stub line 58, which extends between the gas outlet 24 and the gas inlet 34 of the gas delivery device 14, a short-circuiting valve 60 and a microstructured plasma generator 62, through which the breathing gas carried in stub line 58 flows in the self-disinfection operation and which thus enriches this breathing gas with a disinfecting plasma.

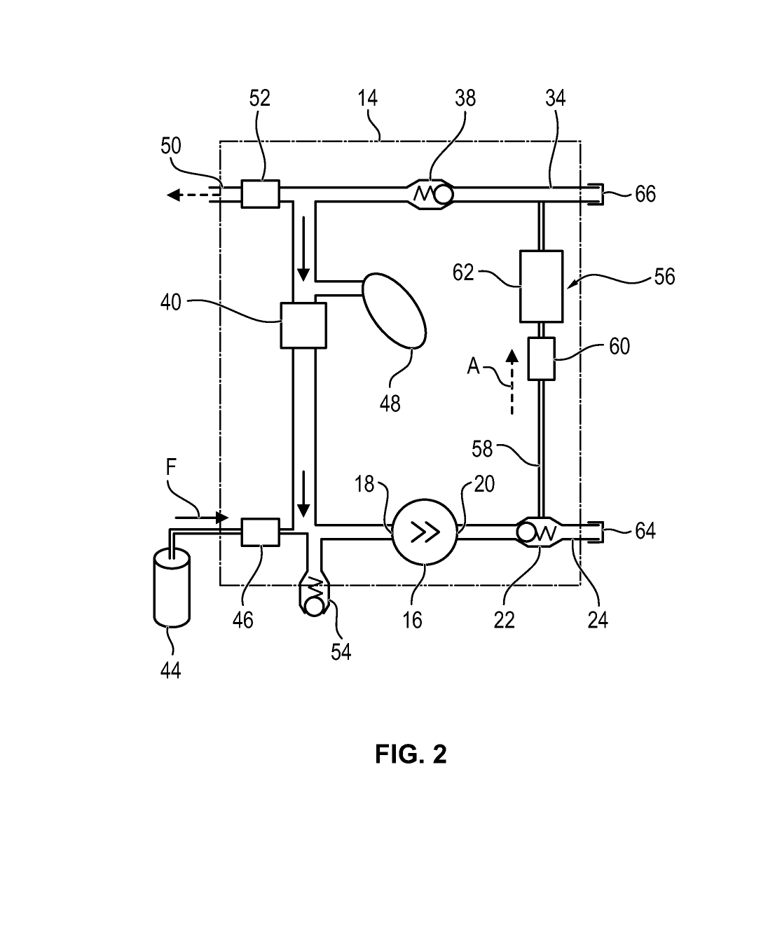

FIG. 2 shows the device 10 in the self-disinfection operation.

The gas outlet 24 and gas inlet 34 of the gas delivery device 14 are closed with a respective blind plug 64 and 66 in the self-disinfection operation. It is thus ensured that the gas delivery device 14 forms a closed-circuit system including the short-circuiting unit 56. Blind plugs 64 and 66 prevent the Y-piece 28 from being coupled in and thus they prevent the normal respiration operation.

As is shown in FIG. 2, the carbon dioxide absorber 42 is removed from the gas delivery device 14 in the self-disinfection operation. The reason for this is, besides an increase in volume, the high moisture content, which is present in the carbon dioxide absorber 42 and counteracts drying and shortens the life of the plasma.

The short-circuiting valve 60 is opened (e.g., by the control unit 88) in the self-disinfection operation, so that the breathing gas stream A is sent through the stub line 58 and is thus enriched with disinfecting plasma, which is generated by the microstructured plasma generator 62. The breathing gas stream A enriched with the plasma is thus sent through all gas-carrying device components of the gas delivery device 14, as a result of which these components are disinfected. Plasma generator 56 is operated cyclically during the self-disinfection operation, and the duration of the cycles is coordinated with the life of the plasma added to the breathing gas stream A.

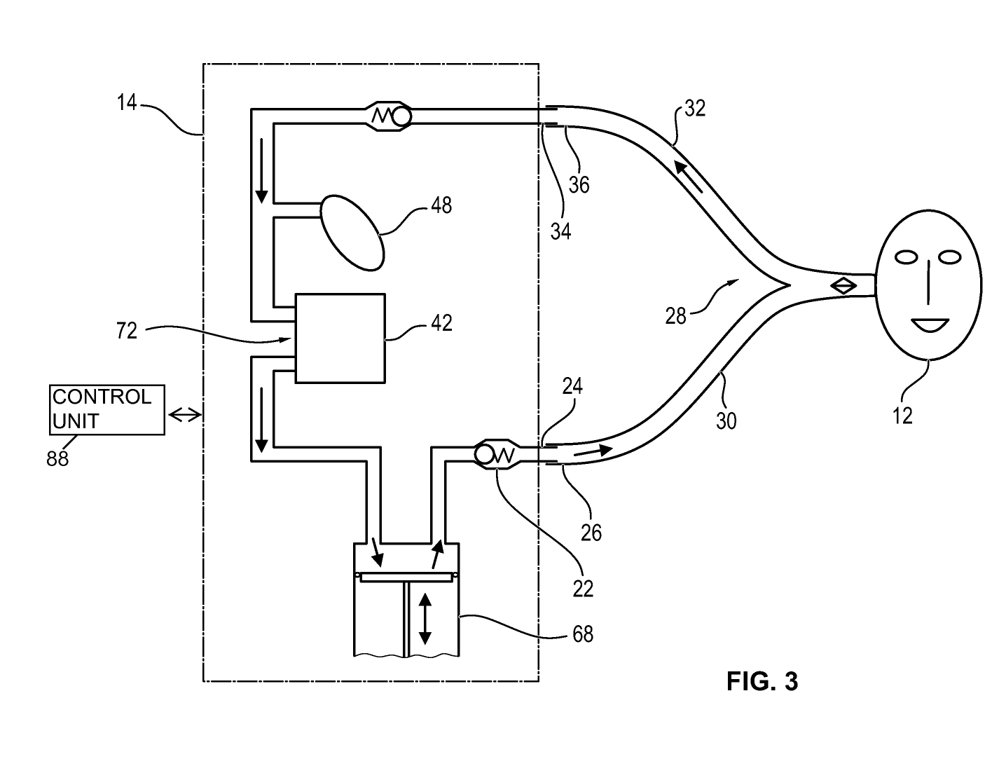

FIGS. 3 through 7 show further exemplary embodiments of the device 10 according to the present invention, and the components that correspond to those of the first exemplary embodiment are designated in these figures with the reference numbers used in FIGS. 1 and 2. For the embodiments of FIGS. 3 through 7 the control unit 88 is operatively connected to gas delivery device 14 as shown only in FIGS. 3, 5 and 6.

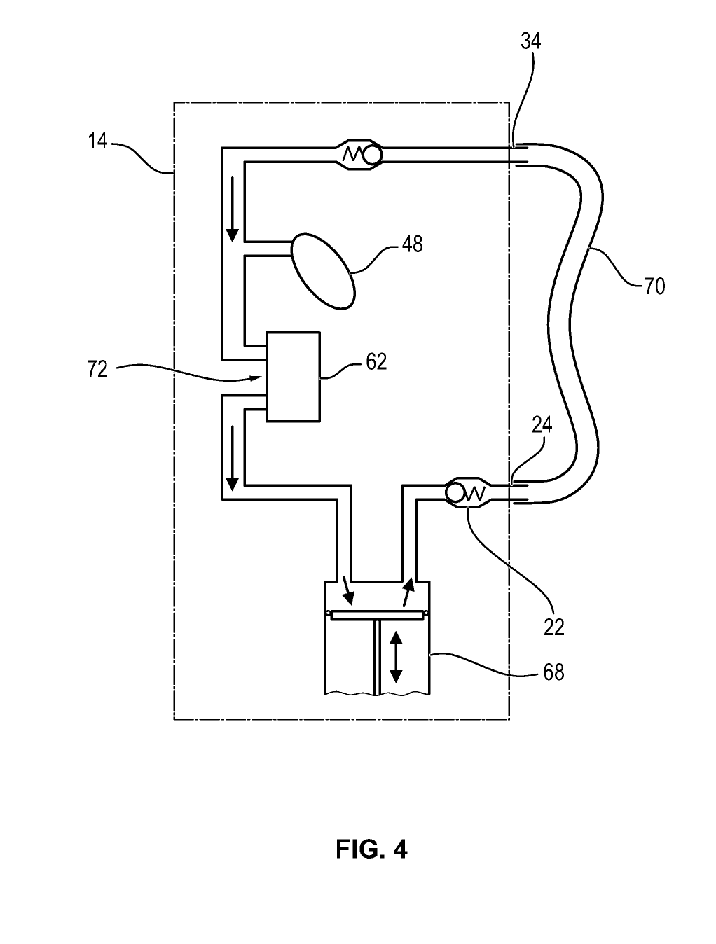

A second exemplary embodiment of the device 10 is shown in the respiration operation in FIG. 3 and in the self-disinfection operation in FIG. 4.

This second exemplary embodiment differs from the first exemplary embodiment according to FIGS. 1 and 2 at first in that a piston and cylinder unit 68 is provided for generating the breathing gas stream A instead of the radial blower 16. Furthermore, as is shown in FIG. 4, the short-circuiting unit 56 is formed in the second exemplary embodiment by a simple tube 70, which is plugged with one of its ends into the gas outlet 24 with its other end into the gas inlet 34. Finally, the gas delivery device 14 in the second exemplary embodiment has a mechanical interface 72, via which the carbon dioxide absorber 42 is coupled into the gas delivery device 14 in the respiration operation and the plasma generator 62 is coupled into the gas delivery device 14 in the self-disinfection operation. The mechanical interface 72 may also include an electrical interface for signals from the control unit 88 to the plasma generator 62.

Consequently, the plasma generator 62 is not installed permanently in the device 10 in the second exemplary embodiment, but a plurality of devices can be used for the hygienic processing.

The second exemplary embodiment otherwise corresponds in its design and mode of operation to the first exemplary embodiment.

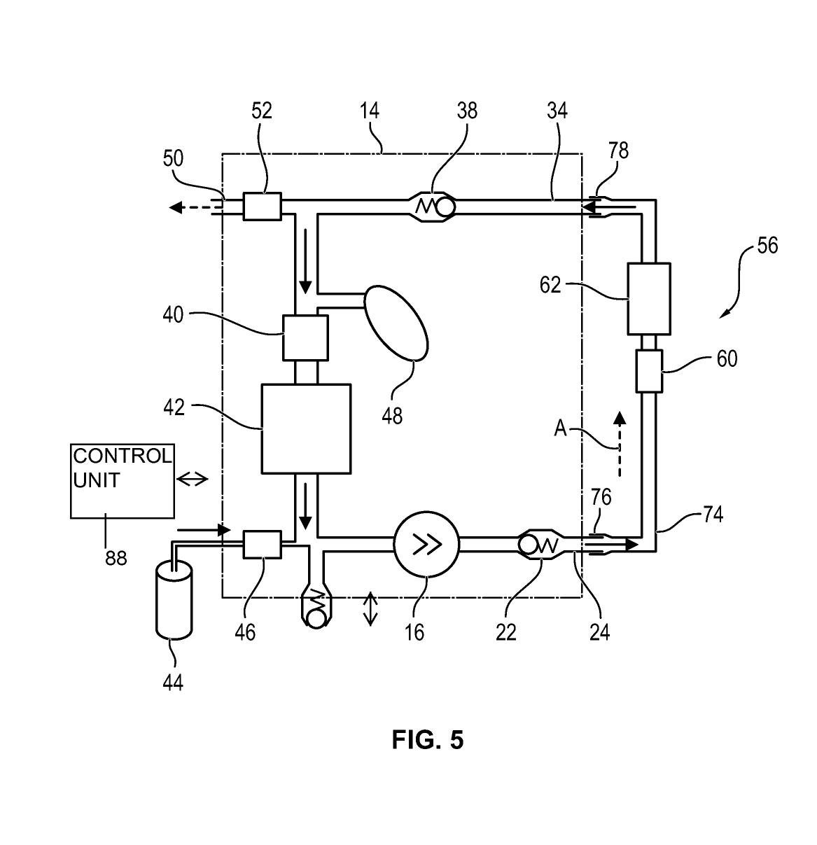

FIG. 5 shows a third exemplary embodiment of the device 10 in the self-disinfection operation.

Unlike in the first exemplary embodiment shown in FIGS. 1 and 2, the short-circuiting unit 56 is not integrated permanently in the gas delivery device 14 in the third exemplary embodiment but is formed from a tube 74 forming a stub line, which tube can be coupled to the gas outlet 24 and gas inlet 34 via two connection parts 76 and 78. The two connection parts 76 and 78 may also include an electrical interface for signals from the control unit 88 to the plasma generator 62.

Just as the second exemplary embodiment shown in FIG. 4, the third exemplary embodiment according to FIG. 5 also has the advantage that the short-circuiting unit 56 makes it impossible to couple the patient 12 to the gas inlet 24 and the gas outlet 34 of the gas delivery device 14 in the self-disinfection operation.

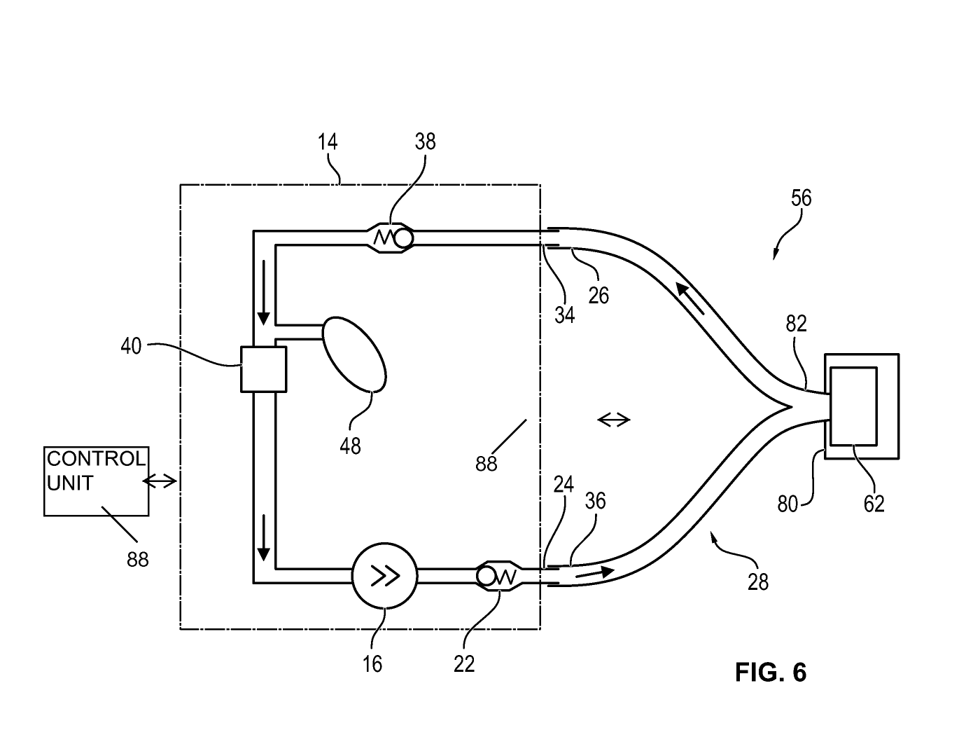

FIG. 6 shows a third exemplary embodiment of the device 10 in the self-disinfection operation.

In the fifth exemplary embodiment, the short-circuiting unit 56 is formed from the Y-piece 28 and a housing 80, which is detachably connected to a third connection part 82 of the Y-piece. This third connection part 82 is used in the normal respiration operation for coupling with the patient. Plasma generator 62 is arranged in housing 80. The Y-piece 28 and the housing 80 may also include an electrical interface and lines for signals from the control unit 88 to the plasma generator 62.

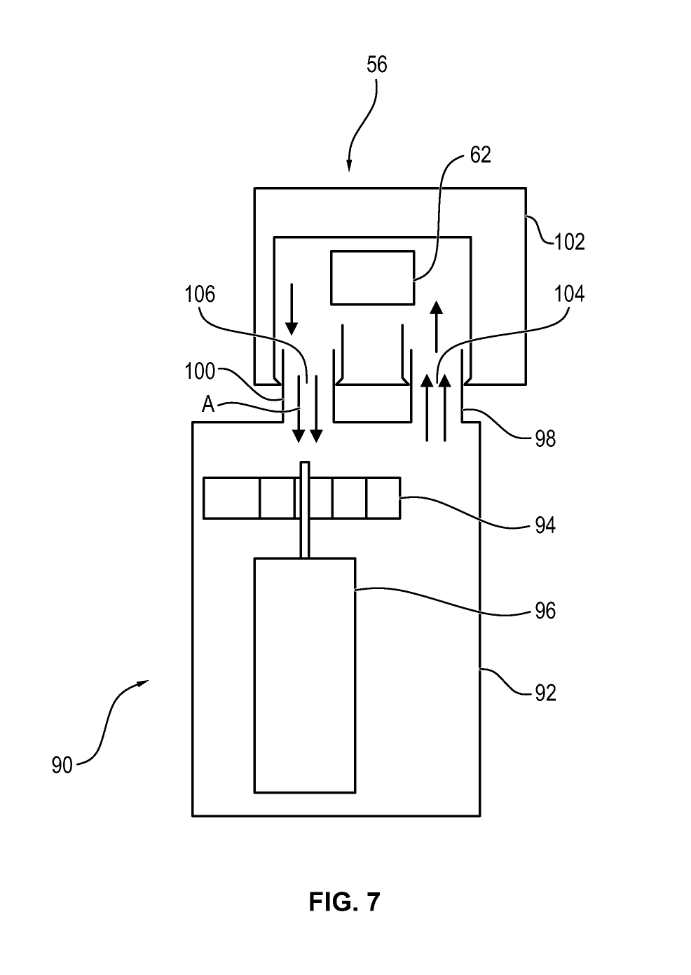

FIG. 7 shows how the present invention can be used for the hygienic processing of an individual device component, namely, a blower unit 90 is itself the mechanical respiration device which is hygienically processed.

Blower unit 90 comprises a blower cartridge 92, in which a blade wheel 94 and a motor 96 rotatingly driving the blade wheel 94 are arranged. Blower cartridge 92 has a gas outlet 98 and a gas inlet 100. Via gas inlet 100, the rotating blade wheel 94 draws the gas stream A into the blower cartridge 92. The gas stream A is again discharged from the blower cartridge 92 from the gas outlet 98.

The short-circuiting unit 56 comprises in this exemplary embodiment a housing 102, in which the plasma generator 62 is located. Housing 102 has a housing inlet 104 that can be coupled with the gas outlet 98 and a gas outlet 106 that can be coupled with the gas inlet 100. In the self-disinfection operation, the plasma generator 62 enriches the gas stream A, which circulates through the arrangement formed from the blower unit 90 and housing 102, with plasma, as a result of which the blower unit 90 is disinfected.

The blower unit 90 shown in FIG. 7 is only an example of a flow module, which can be hygienically processed in the manner according to the present invention. It is also possible, for example, to disinfect the piston and cylinder unit shown in FIG. 3 in the manner shown in FIG. 7.

While specific embodiments of the invention have been shown and described in detail to illustrate the application of the principles of the invention, it will be understood that the invention may be embodied otherwise without departing from such principles.

APPENDIX
LIST OF REFERENCE NUMBERS

| | |
|---|---|
| 10 | Device |
| 12 | Patient |
| 14 | Gas delivery device |
| 16 | Blower |
| 18 | Gas inlet of blower |
| 20 | Gas outlet of blower |
| 22 | Inspiration valve |
| 24 | Gas outlet |
| 26 | Connection piece |
| 28 | Y-piece |
| 30 | Inspiration section |
| 32 | Expiration section |
| 34 | Gas inlet |
| 36 | Connection piece |
| 38 | Expiration valve |
| 40 | Heater |
| 42 | Carbon dioxide absorber |
| 44 | Fresh air feeding device |
| 46 | Fresh air valve |
| 48 | Manual bag |
| 50 | Anesthetic gas escape line |
| 52 | Valve |
| 54 | Emergency valve |
| 56 | Short-circuiting unit |
| 58 | Stub line |
| 60 | Short-circuiting valve |
| 62 | Plasma generator |
| 64, 66 | Blind plug |
| 68 | Piston and cylinder unit |
| 70 | Tube |
| 72 | Mechanical interface |
| 74 | Tube |
| 76, 78 | Connection parts |
| 80 | Housing |
| 82 | Connection part |
| 90 | Blower unit |
| 92 | Blower cartridge |
| 94 | Blade wheel |
| 96 | Motor |
| 98 | Gas outlet |
| 100 | Gas inlet |
| 102 | Housing |
| 104 | Housing inlet |
| 106 | Housing outlet |
| A | Breathing gas stream |
| F | Fresh air |

What is claimed is:

1. A mechanical respiration device for the mechanical respiration of a patient, the mechanical respiration device comprising:
a gas delivery device comprising a flow module for generating a gas stream, a gas outlet and a gas inlet and a gas-carrying short-circuiting unit coupling said gas outlet and said gas inlet with one another for short-circuiting said gas delivery device; and
a plasma generator operatively connected to said short-circuited gas delivery device for enriching a gas stream generated in said short-circuited gas delivery device by said flow module with a disinfecting plasma for the hygienic treatment of the device, wherein the conduction of a disinfecting process and the validation of the process is controlled by the device itself.

2. A mechanical respiration device in accordance with claim 1, wherein said plasma generator is integrated in said gas-carrying short-circuiting unit.

3. A mechanical respiration device in accordance with claim 1, wherein said gas-carrying short-circuiting unit comprises a gas-carrying stub line, which connects said gas outlet to said gas inlet of said gas delivery device, and a controllable short-circuiting valve for opening said stub line.

4. A mechanical respiration device in accordance with claim 3, wherein said stub line is formed from a tube with two connection parts, of which a first connection part is attachable to said gas outlet and a second connection part is attachable to said gas inlet.

5. A mechanical respiration device in accordance with claim 1, wherein said gas-carrying short-circuiting unit comprises a Y-piece with three connection parts, of which a first connection part is coupleable with said gas outlet, a second connection part is coupleable with said gas inlet and a third connection part is coupleable with said plasma generator.

6. A mechanical respiration device in accordance with claim 1, further comprising gas-carrying device component, wherein said gas delivery device has a mechanical interface for optionally coupling and uncoupling a gas-carrying device component or coupling and uncoupling said plasma generator.

7. A mechanical respiration device in accordance with claim 6, wherein said gas-carrying device component is a carbon dioxide absorber.

8. A mechanical respiration device in accordance with claim 1, wherein said gas delivery device has a heater for heating the gas stream enriched with the disinfecting plasma.

9. A mechanical respiration device in accordance with claim 1, wherein said flow module is a blower.

10. A mechanical respiration device in accordance with claim 1, wherein said flow module is a piston and cylinder unit.

11. A mechanical respiration device in accordance with claim 1, wherein said gas outlet is associated with an inspiration branch and said gas inlet is associated with an expiration branch.

12. A mechanical respiration device in accordance with claim 1, wherein said gas outlet and said gas inlet are formed at the flow module.

13. A mechanical respiration device in accordance with claim 12, wherein said gas-carrying short-circuiting unit is formed from a housing, in which said plasma generator is accommodated and which has a housing inlet coupleable with a gas outlet of said flow module and a housing outlet coupleable with a gas inlet of said flow module.

14. A process for the hygienic processing of a mechanical respiration device intended for the mechanical respiration of a patient, the process comprising the steps of:
providing the mechanical respiration device for the mechanical respiration of a patient with a gas delivery device comprising a flow module for generating a gas stream, a gas outlet and a gas inlet and a gas-carrying short-circuiting unit coupling the gas outlet and the gas inlet with one another for short-circuiting the gas delivery device and a plasma generator operatively connected to the short-circuited gas delivery device for enriching a gas stream generated in the short-circuited gas delivery device by the flow module with a disinfecting plasma;
short-circuiting the gas delivery device by means of the gas-carrying short-circuiting unit, which couples with the gas outlet and the gas inlet of the gas delivery device with one another;
generating a gas stream by means of the flow module in the short-circuited gas delivery device; and
enriching the gas stream generated in the short-circuited gas delivery device by the flow module with a disinfecting plasma by means of the plasma generator coupled into the short-circuited gas delivery device, wherein the conduction of the process and the validation of the processing can be controlled by the device itself.

15. A process in accordance with claim 14, wherein the plasma generator is cyclically put into operation.

16. A process in accordance with claim 14, wherein a part of the gas stream generated in the short-circuited gas delivery device is replaced with fresh gas.

17. A mechanical respiration device for the mechanical respiration of a patient, the mechanical respiration device comprising:
a gas delivery device comprising a flow module for generating a gas stream, a gas outlet and a gas inlet and a gas-carrying short-circuiting unit coupling said gas outlet and said gas inlet with one another for short-circuiting said gas delivery device;
a control unit controlling the gas delivery device; and
a plasma generator operatively connected to said short-circuited gas delivery device for enriching a gas stream generated in said short-circuited gas delivery device by said flow module with a disinfecting plasma for the hygienic treatment of the device, wherein the control unit controls the gas delivery device and the plasma generator during a disinfecting process and provides a validation of the process.

18. A mechanical respiration device in accordance with claim 17, wherein said plasma generator is integrated in said gas-carrying short-circuiting unit.

19. A mechanical respiration device in accordance with claim 17, wherein said gas-carrying short-circuiting unit comprises a gas-carrying stub line, which connects said gas outlet to said gas inlet of said gas delivery device, and a controllable short-circuiting valve for opening said stub line.

20. A mechanical respiration device in accordance with claim 19, wherein said stub line is formed from a tube with two connection parts, of which a first connection part is attachable to said gas outlet and a second connection part is attachable to said gas inlet.
